(19) United States
(12) Patent Application Publication
(10) Pub. No.: US 2014/0235685 A1
(43) Pub. Date: Aug. 21, 2014

(54) PHARMACEUTICAL COMPOSITIONS COMPRISING 4-BROMO-N-(IMIDAZOLIDIN-2-YLIDENE)-1H-BENZIMIDAZOL-5-AMINE FOR TREATING SKIN DISEASES

(71) Applicant: Allergan, Inc., Irvine, CA (US)

(72) Inventors: Mohammed I. Dibas, Laguna Niguel, CA (US); John E. Donello, Dana Point, CA (US); Daniel W. Gil, Corona Del Mar, CA (US)

(73) Assignee: Allergan, Inc., Irvine, CA (US)

(21) Appl. No.: 14/225,071

(22) Filed: Mar. 25, 2014

Related U.S. Application Data
(63) Continuation of application No. 13/551,930, filed on Jul. 18, 2012.
(60) Provisional application No. 61/510,708, filed on Jul. 22, 2011.

(51) Int. Cl. A61K 31/4184

(52) U.S. Cl. CPC A61K 31/4184
USPC 514/392

(57) ABSTRACT

The present invention relates to a method for treating skin diseases in a patient in need thereof which comprises of administering a pharmaceutical composition comprising a therapeutically effective amount of 4-bromo-N-(imidazolidin-2-ylidene)-1H-benzimidazol-5-amine or a pharmaceutically acceptable salt thereof and one or more pharmaceutically acceptable excipients.

### CROSS-REFERENCE TO RELATED APPLICATIONS

This patent application is a continuation of U.S. Non Provisional application Ser. No. 13/551,930, filed on Jul. 18, 2012 which claims the benefit of U.S. Provisional Application Ser. No. 61/510,708, filed on Jul. 22, 2011, which are incorporated by reference herein in their entirety.

### FIELD OF THE INVENTION

The present invention relates to a method for treating skin diseases in a patient in need thereof which comprises administering a pharmaceutical composition comprising a therapeutically effective amount of 4-bromo-N-(imidazolidin-2-ylidene)-1H-benzimidazol-5-amine or a pharmaceutically acceptable salt thereof and one or more pharmaceutically acceptable excipients, carriers or diluents.

### BACKGROUND OF THE INVENTION

Three alpha 1 and three alpha 2 adrenergic receptors have been characterized by molecular and pharmacological methods. Activation of these alpha 2 receptors evokes physiological responses having useful therapeutic actions. Alpha adrenergic agonists act on the peripheral vasculature to cause vasoconstriction and thereby ameliorate the symptoms of inflammatory skin disorders. Alpha adrenergic agonists minimize redness on ocular mucosal tissue to treat conjunctival redness, for nasal mucosa, as a decongestant for the treatment of allergic rhinitis, and for rectal mucosal administration suitable for curing hemorrhoids.

H. E. Baldwin describes the diagnosis and the actual treatments of rosacea and related skin diseases, in the Journal of Drugs in Dermatology 2012, Vol. 11(6) pages 725-730.

U.S. Pat. No. 6,680,062 discloses topical cosmetic and pharmaceutical compositions for the treatment of the skin.

U.S. Patent Application Publication No. 2012/0035123 describes combinations of compounds for treating skin diseases.

U.S. Pat. No. 7,812,049 discloses a method for treating erythema resulting from rosacea comprising oxymetazoline. Oxymetazoline is a selective alpha-1 agonist and partial alpha-2 agonist topical decongestant.

Compound 4-bromo-N-(imidazolidin-2-ylidene)-1H-benzimidazol-5-amine is known as a potent alpha 2 adrenergic receptor pan agonist, activating all three alpha-2 receptor subtypes.

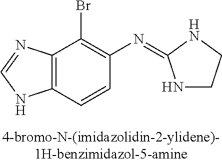 [SMILES: Brc1c(N=C2NCCN2)ccc2[nH]cnc12]

4-bromo-N-(imidazolidin-2-ylidene)-1H-benzimidazol-5-amine is disclosed in U.S. Pat. No. 6,316,637, and may be prepared according to the disclosure of U.S. Pat. No. 6,495,583 B1; both patents are hereby incorporated by reference in their entirety.

### SUMMARY OF THE INVENTION

We have now discovered that the pharmaceutical compositions of 4-bromo-N-(imidazolidin-2-ylidene)-1H-benzimidazol-5-amine are useful for the treatment of skin diseases. The present invention relates to pharmaceutical compositions containing as active ingredient 4-bromo-N-(imidazolidin-2-ylidene)-1H-benzimidazol-5-amine for treatment of skin diseases.

In another aspect the present invention relates to a method for treating skin diseases in a patient in need thereof which comprises administering a pharmaceutical composition comprising a therapeutically effective amount of 4-bromo-N-(imidazolidin-2-ylidene)-1H-benzimidazol-5-amine or a pharmaceutically acceptable salt thereof.

In another aspect the present invention relates to a method for improving skin diseases in a patient in need thereof which comprises administering a pharmaceutical composition comprising a therapeutically effective amount of 4-bromo-N-(imidazolidin-2-ylidene)-1H-benzimidazol-5-amine or a pharmaceutically acceptable salt thereof.

The compound may be administered through different routes, including but not limited to topical dermatological application of an effective dose, direct injection, or formulations that may further enhance the long duration of actions such as a slow releasing pellet, suspension, gel, solution, cream, ointment, foams, emulsions, microemulsions, milks, serums, aerosols, sprays, dispersions, microcapsules, vesicles, microparticles, wet cloths, dry cloths, facial cloths, or sustained delivery devices such as any suitable drug delivery system known in the art.

### BRIEF DESCRIPTION OF THE DRAWINGS

FIG. 1 shows that topical application to the skin of compound 4-bromo-N-(imidazolidin-2-ylidene)-1H-benzimidazol-5-amine, at a concentration of 0.5%, inhibited the induced vessel dilation caused at 37° C.

FIG. 2 shows cumulative amount of drug in receptor solution at 48 hrs.

### DETAILED DESCRIPTION OF THE INVENTION

In one aspect of the invention, there is provided a method for treating skin diseases in a patient in need thereof which comprises, consists essentially of or consists of administering an amount of a pharmaceutical composition comprising, consisting essentially of or consisting of a therapeutically effective amount of 4-bromo-N-(imidazolidin-2-ylidene)-1H-benzimidazol-5-amine or a pharmaceutically acceptable salt thereof. By “skin diseases” it should be understood any condition, complaint or affliction associated with the listed diseases.

Skin diseases which may be treated with pharmaceutical compositions containing as active ingredient 4-bromo-N-(imidazolidin-2-ylidene)-1H-benzimidazol-5-amine include, but are not limited to: rosacea, rosacea fulminans, sunburn, psoriasis, menopause-associated hot flashes, flushing and redness associated with hot flashes, erythema associated with hot flashes, hot flashes resulting from orchiectomyatopic dermatitis, treatment of redness and itch from insect bites, photoaging, seborrheic dermatitis, acne, allergic dermatitis, telangiectasia (dilations of previously existing small blood vessels) of the face, angioectasias, rhinophyma (hypertrophy of the nose with follicular dilation), acne-like skin eruptions (may ooze or crust), burning or stinging sensation, erythema of the skin, cutaneous hyperactivity with dilation of blood vessels of the skin, Lyell's syndrome, Stevens-Johnson syndrome, local itching and discomfort associated with hemorrhoids, hemorrhoids, erythema multiforme minor, erythema multiforme major, erythema nodosum, eye puffiness, urticaria, pruritis, purpura, varicose veins, contact dermatitis, atopic dermatitis, nummular dermatitis, generalized exfoliative dermatitis, stasis dermatitis, lichen simplex chronicus, perioral dermatitis, pseudofolliculitis barbae, granuloma annulare, actinic keratosis, basal cell carcinoma, squamous cell carcinoma, eczema.

Skin conditions which result in rosacea can be induced by intake of spicy food, of alcohol, of chocolate, of hot or alcoholic drinks, temperature variations, heat, exposure to ultraviolet or infrared radiation, exposure to low relative humidity, exposure of the skin to strong winds or currents of air, exposure of the skin to surfactants, irritants, irritant dermatological topical agents, and cosmetics or psychological stress.

In one aspect of the invention, there is provided a method for treating ocular diseases in a patient in need thereof which comprises, consists essentially of or consists of administering an amount of a pharmaceutical composition comprising, consisting essentially of or consisting of a therapeutically effective amount of 4-bromo-N-(imidazolidin-2-ylidene)-1H-benzimidazol-5-amine or a pharmaceutically acceptable salt thereof.

Ocular diseases which may be treated with pharmaceutical compositions containing as active ingredient 4-bromo-N-(imidazolidin-2-ylidene)-1H-benzimidazol-5-amine include, but are not limited to: ocular rosacea, subconjuctival hemorrhage, keratitis, herpetic dendritic corneal ulcer, palpebral conjunctiva, chemosis, trachoma cicatrical ptosis, vernal conjunctivitis, symblepharon, pterygium, pterygium postsurgical papilloma, limbal keratoconjunctivitis, episcleritis, scleritis, ocular vein varicosities, Sturge-Weber syndrome, carotid cavernous fistula, conjunctival metaplasia in ectropion.

The actual amount of the compound to be administered in any given case will be determined by a physician taking into account the relevant circumstances, such as the severity of the condition, the age and weight of the patient, the patient's general physical condition, the cause of the condition, and the route of administration.

In another aspect of the invention, there is provided a method for treating skin diseases wherein the pharmaceutical composition comprising, consisting essentially of or consisting of a therapeutically effective amount of 4-bromo-N-(imidazolidin-2-ylidene)-1H-benzimidazol-5-amine, is selected from topical skin application comprising suspensions, gels, solutions, creams, lotions, ointments, foams, emulsions, microemulsions, milks, serums, aerosols, sprays, dispersions, microcapsules, vesicles, microparticles, wet cloths, dry cloths, facial cloths, applications and formulations that may further enhance the long duration of actions such as a slow releasing pellets, direct injection, or sustained delivery devices such as any suitable drug delivery systems known in the art. Pharmaceutical compositions of the present invention can be used for the topical administration including solutions, gels, lotions creams, ointments, foams, mousses, emulsions, microemulsions, milks, serums, aerosols, sprays, dispersions, patches, micelles, liposomes, microcapsules, vesicles and microparticles thereof.

Emulsions, such as creams and lotions that can be used as topical carriers and their preparation are disclosed in Remington: The Science and Practice of Pharmacy 282-291 (Alfonso R. Gennaro Ed. 19^{th} ed. 1995) hereby incorporated herein by reference.

Suitable gels for use in the invention are disclosed in Remington: The Science and Practice of Pharmacy 1517-1518 (Alfonso R. Gennaro Ed. 19^{th} ed. 1995) hereby incorporated herein by reference. Other suitable gels for use within the invention are disclosed in U.S. Pat. No. 6,387,383, U.S. Pat. No. 6,517,847 and U.S. Pat. No. 6,468,989.

In another aspect of the invention, there is provided a method for improving skin diseases including but not limited to: rosacea, rosacea fulminans, sunburn, psoriasis, menopause-associated hot flashes, hot flashes resulting from orchiectomyatopic dermatitis, photoaging, seborrheic dermatitis, acne, allergic dermatitis, telangiectasia (dilations of previously existing small blood vessels) of the face, angioectasias, rhinophyma (hypertrophy of the nose with follicular dilation), acne-like skin eruptions (may ooze or crust), burning or stinging sensation, erythema of the skin, cutaneous hyperactivity with dilation of blood vessels of the skin, Lyell's syndrome, Stevens-Johnson syndrome, local itching and discomfort associated with hemorrhoids, hemorrhoids, erythema multiforme minor, erythema multiforme major, erythema nodosum, eye puffiness, urticaria, pruritis, purpura, varicose veins, contact dermatitis, atopic dermatitis, nummular dermatitis, generalized exfoliative dermatitis, stasis dermatitis, lichen simplex chronicus, perioral dermatitis, pseudofolliculitis barbae, granuloma annulare, actinic keratosis, basal cell carcinoma, squamous cell carcinoma, eczema.

In another aspect of the invention, there is provided a method of decreasing the irritation of skin associated with rosacea treatment regimen of topically applied a therapeutically effective amount of 4-bromo-N-(imidazolidin-2-ylidene)-1H-benzimidazol-5-amine, the method of treating telangiectasia or angioectasias with a therapeutically effective amount of 4-bromo-N-(imidazolidin-2-ylidene)-1H-benzimidazol-5-amine, and therefore, it also includes the method of reducing redness associated with the appearance of rosacea.

In another aspect of the invention, there is provided a method for treating skin diseases including but not limited to: rosacea induced by intake of spicy food, chocolate, alcohol, hot or alcoholic drinks, temperature variations, heat, exposure to ultraviolet or infrared radiation, exposure to low relative humidity, exposure of the skin to strong winds or currents of air, exposure of the skin to surfactants, irritants, irritant dermatological topical agents, and cosmetics or psychological stress.

In another aspect of the invention, there is provided an article of manufacture comprising packaging material and a pharmaceutical agent contained within said packaging material, wherein the pharmaceutical agent is therapeutically effective for treating a skin disease and wherein the packaging material comprises a label which indicates the pharmaceutical agent can be used for treating a skin disease and wherein said pharmaceutical agent comprises an effective amount of 4-bromo-N-(imidazolidin-2-ylidene)-1H-benzimidazol-5-amine or a salt thereof.

“Pharmaceutical composition,” as used here, means a composition that is suitable for administering to human patients for disease treatment. In one embodiment the compound of the invention is formulated as a pharmaceutically acceptable salt which further includes one or more organic or inorganic carriers or excipients suitable for dermatological applications. The pharmaceutically acceptable excipients may include one or more skin-penetrating agents, moisturizers, preservatives, gelling agents, protective agents, oil-in-water, water-in-oil, water-in-oil-in-water, and oil-in-water-in-silicon emulsions. The pharmaceutical composition may comprise excipients, binders, lubricants, solvents, disintegrants, or enhancers of cutenous penetration.

“Pharmaceutically acceptable salt” refers to those salts which retain the biological effectiveness and properties of the free base and which are obtained by reaction with inorganic acids such as hydrochloric acid, hydrobromic acid, sulfuric acid, nitric acid, phosphoric acid, or an organic acid such as for example, acetic acid, hydroxyacetic acid, propanoic acid, lactic acid, pyruvic acid, malonic acid, fumaric acid, maleic acid, oxalic acid, tartaric acid, succinic acid, malic acid, ascorbic acid, benzoic acid, tannic acid, pamoic acid, citric acid, methylsulfonic acid, ethanesulfonic acid, benzenesulfonic acid, formic and, salicylic acid and the like (Handbook of Pharmaceutical Salts, P. Heinrich Stahal& Camille G. Wermuth (Eds), Verlag Helvetica Chemica Acta-Zürich, 2002, 329-345). Compound 4-bromo-N-(imidazolidin-2-ylidene)-1H-benzimidazol-5-amine may be formulated with efficacy enhancing components as disclosed in U.S. Pat. No. 7,491,383 B2, which is hereby incorporated by reference in its entirety.

Compound 4-bromo-N-(imidazolidin-2-ylidene)-1H-benzimidazol-5-amine has physiochemical and pharmacokinetic properties that are beneficial for sustained activity, particularly when the drug is delivered continuously (e.g. to the skin by a dermal patch). The present invention may be used in conjunction with rosacea treatments of topically applied such as macrocyclic lactones of the avermectin family, macrolides known as milbemycins, other alpha 1 or alpha 2 receptor agonists, retinoids, phytoshingosine, green tea extract, azaleic acid.

The present invention may also be used in conjunction with other classes of compounds such as:
Antimicrobials (such as antiparasitic, antibacterial, antifungal, antiviral);
Metronidazole, ivermectin, clindamycin, erythromycin, tetracycline, doxycycline, minocycline;
Steroidal and non-steroidal anti-inflammatory agents (such as corticosteroids, tacrolimus, pimecrolimus, cyclosporine A);
Antiangiogenesis agents;
Sunscreens or anything that functions like a sunscreen (such as titanium dioxide, zinc oxide, avobenzone);
Antioxidants (such as Vitamins C, E, quercetin, resveratrol);
Other alpha agonists (such as brimonidine, oxymetazoline, clonidine);
Beta blockers (such as nadolol, propanolol, carvedilol);
Antihistamines;
Retinoids (such as tretinoin, adapalene, tazarotene, isotretinoin, retinaldehyde) Benzoyl peroxide;
Menthol and other “cooling” agents;
Sodium sulfacetamide and derivatives;
Serine protease (kallikrein) inhibitors (such as aminocaproic acid).

The present invention is not to be limited in scope by the exemplified embodiments, which are only intended as illustrations of specific aspects of the invention. Various modifications of the invention, in addition to those disclosed herein, will be apparent to those skilled in the art by a careful reading of the specification, including the claims, as originally filed. It is intended that all such modifications will fall within the scope of the appended claims.

### Example 1

### Rat Blood Flow Assay

### Background

α-adrenergic agonists that act on the sympathetic nervous system outflow can regulate cutaneous blood flow in response to temperature changes.

### Method

A laser Doppler microvascular perfusion monitor (Laser Doppler Flowmetry LDP technique), was used to monitor red blood cell perfusion in the microvasculature of the hind foot pad. The laser doppler flowmetry (LDP) is an OxyFlo Microvascular Perfusion Monitor, from Oxford Optronix LTd. UK.

Briefly, 15 μL of test articles were applied topically to one hind foot pad of anaesthetized CD rats and 15 μL of vehicle was applied to the other footpad. The hairless CD rats used are a spontaneous mutation model isolated from a Crl:CD(SD) colony in Charles River, in the late 1980s. Rederived in 1993 and subsequently transferred to Charles River, Raleigh, N.C. for barrier room production. The model does not exhibit the typical characteristics of hair growth and loss found in other hairless models. Specific genetic analysis to identify the mutation has not been undertaken. Histopathology has determined the model is euthymic.

Thirty minutes following test article administration, dynamic blood flow changes were measured and recorded every 15 seconds for 4 minutes per temperature interval for 5 intervals (22° C.→37° C.→4° C.→37° C.→22° C.). Rats were placed on a 37° C. heat pad to increase their temperature and on an ice pad to decrease their temperature to 4° C. The level of blood flow in the two paws was compared. Compound 4-bromo-N-(imidazolidin-2-ylidene)-1H-benzimidazol-5-amine showed 17% overall blood flow inhibition. BPU Flow 2-Sample T-Test Unequal Variance showed P=0.014 (<0.05). It has a significant difference.

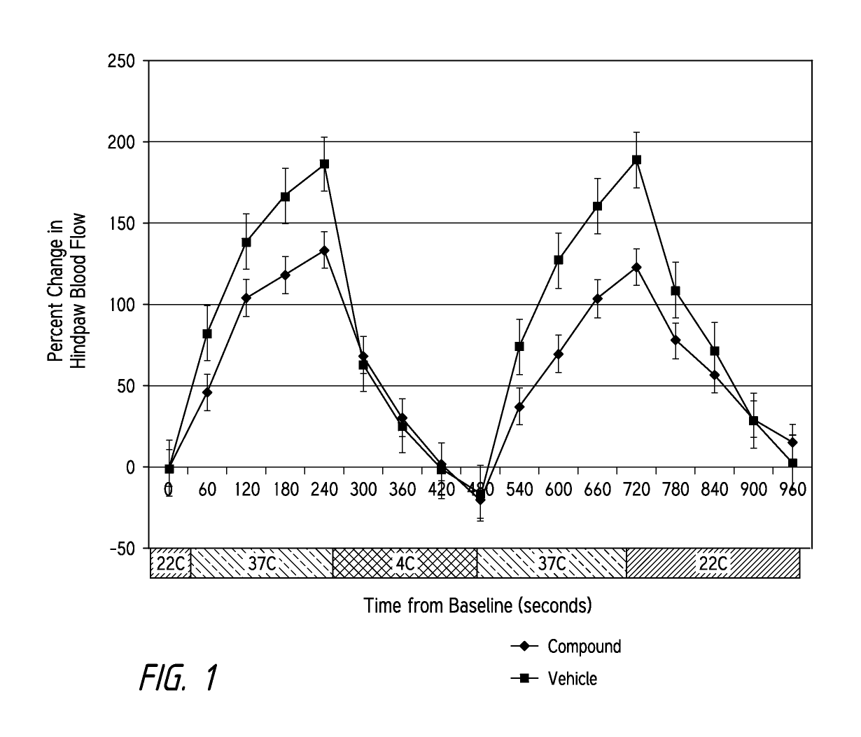

FIG. 1 showed that topical application to the skin of compound 4-bromo-N-(imidazolidin-2-ylidene)-1H-benzimidazol-5-amine, at a concentration of 0.5%, inhibited the induced vessel dilation caused at 37° C.

### Example 2

### In Vitro Human Skin Permeability Assay

Human, ex vivo, trunk skin was cut into multiple smaller sections large enough to fit on nominal 1 cm² or 2 cm² static Franz diffusion cells. The dermal receptor compartment was filled to capacity with receptor solution consisting of 1×PBS with 0.008% Gentamicin, and the epidermal chamber (chimney) is left open to ambient laboratory environment. The cells were placed in a diffusion apparatus in which the receptor solution in contact with the underside of the dermis was stirred magnetically at ˜600 RPM and its temperature maintained to achieve a skin surface temperature of 32.0±1.0° C.

To assure the integrity of each skin section, its permeability to tritiated water was determined before application of the test products. Skin specimens in which absorption of ³H₂O was less than 1.56 μL-equ/cm² were considered acceptable.

Test products were applied to three (3) replicate sections of the same skin donor for each donor, evaluating three (3) donors for the designated dose duration. A dose of 5 mg formulation/cm²/skin section was evenly dispersed and rubbed into the skin surface using a glass rod.

At designated time points and at the end of the study dose duration, the receptor solution was removed in its entirety, and a predetermined volume aliquot was saved for subsequent analysis. After the last receptor sample was collected, the donor compartment (chimney) was removed, and the surface of the skin was cleansed twice to collect any non-absorbed formulation from the skin surface. Following the surface cleanse, the skin was tape stripped to remove the stratum corneum. The tape strips were extracted overnight in acetonitrile and analyzed for compound content. The skin was then removed from the diffusion cell, split into epidermis and dermis, and each skin sample extracted overnight in an appropriate solvent, and analyzed for content of the drug of interest. All samples were stored at −20° C. (±10° C.) pending analysis. Concentrations of each of the compounds of interest were quantified using an HPLC/MS analytical method.

Replicates within donors were averaged and the standard deviation calculated for each key parameter. Within donor averages were then collated and the across donor population mean with standard error was calculated.

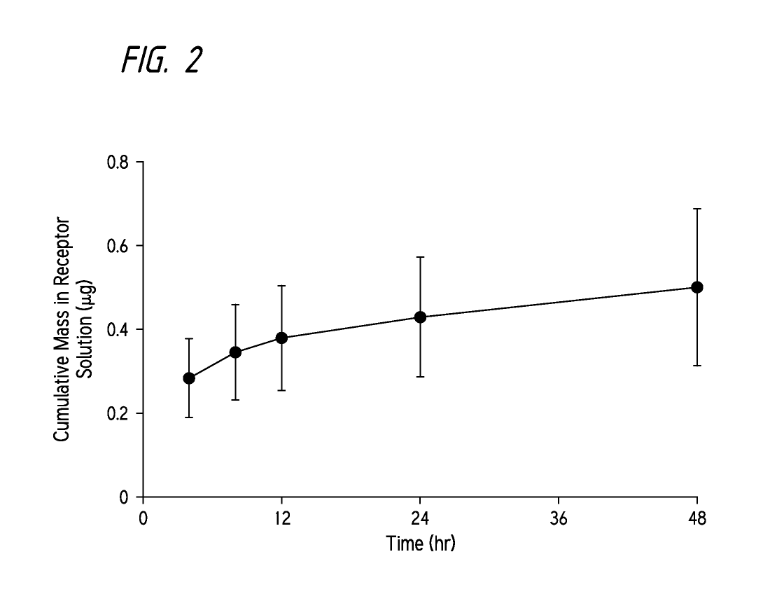

FIG. 2 shows the cumulative percutaneous absorption of 4-bromo-N-(imidazolidin-2-ylidene)-1H-benzimidazol-5-amine that appears in the receptor solution under the skin after a 0.54% (w/v) dose is applied topically to the skin.

What is claimed is:

1. A method for treating skin diseases in a patient suffering thereof, which comprises treating said patient with a pharmaceutical composition comprising a therapeutically effective amount of 4-bromo-N-(imidazolidin-2-ylidene)-1H-benzimidazol-5-amine or a salt thereof.

2. The method according to claim 1, wherein the disease is selected from: rosacea, rosacea fulminans, sunburn, psoriasis, menopause-associated hot flashes, hot flashes resulting from orchiectomyatopic dermatitis, photoaging, seborrheic dermatitis, acne, allergic dermatitis, telangiectasia of the face, angioectasias, rhinophyma, acne-like skin eruptions, burning or stinging sensation, erythema of the skin, cutaneous hyperactivity with dilation of blood vessels of the skin, Lyell's syndrome, Stevens-Johnson syndrome, local itching and discomfort associated with hemorrhoids, hemorrhoids, erythema multiforme minor, erythema multiforme major, erythema nodosum, eye puffiness, urticaria, pruritis, purpura, varicose veins, contact dermatitis, atopic dermatitis, nummular dermatitis, generalized exfoliative dermatitis, stasis dermatitis, lichen simplex chronicus, perioral dermatitis, pseudofolliculitis barbae, granuloma annulare, actinic keratosis, basal cell carcinoma, squamous cell carcinoma, eczema, ocular rosacea, subconjuctival hemorrhage, keratitis, herpetic dendritic corneal ulcer, palpebral conjunctiva, chemosis, trachoma cicatrical ptosis, vernal conjunctivitis, symblepharon, pterygium, pterygium postsurgical papilloma, limbal keratoconjunctivitis, episcleritis, scleritis, ocular vein varicosities, Sturge-Weber syndrome, carotid cavernous fistula, conjunctival metaplasia in ectropion.

3. The method according to claim 1, wherein the disease is rosacea.

4. The method according to claim 1, wherein the disease is psoriasis.

5. The method according to claim 1, wherein the disease is rosacea fulminans.

6. The method according to claim 1, wherein the disease is telangiectasia of the face.

7. The method according to claim 1, wherein the disease is erythema of the skin.

8. The method according to claim 1, wherein the pharmaceutical composition further includes one or more pharmaceutically acceptable excipients.

9. The method according to claim 1, wherein the pharmaceutical composition is administered topically to the skin.

10. The method according to claim 1, wherein the composition is selected from suspensions, gels, solutions, creams, lotions, ointments, foams, emulsions, microemulsions, milks, serums, aerosols, sprays, dispersions, microcapsules, vesicles, microparticles, wet cloths, dry cloths, facial cloths, applications and formulations that may further enhance the long duration of actions such as a slow releasing pellet.

11. The method according to claim 1, wherein the composition is a cream.

12. The method according to claim 1, wherein the composition is a gel.

13. The method according to claim 1, wherein the composition is a lotion.

14. The method according to claim 1, wherein the composition is part of a facial cloth.

15. An article of manufacture comprising packaging material and a pharmaceutical agent contained within said packaging material, wherein the pharmaceutical agent is therapeutically effective for treating a skin disease and wherein the packaging material comprises a label which indicates the pharmaceutical agent can be used for treating a skin disease and wherein said pharmaceutical agent comprises an effective amount of 4-bromo-N-(imidazolidin-2-ylidene)-1H-benzimidazol-5-amine or a salt thereof.
